(19) United States
(12) Patent Application Publication
(10) Pub. No.: US 2009/0238788 A1
(43) Pub. Date: Sep. 24, 2009

(54) DELIVERY OF HYDROGELS AS SPRAYS

(75) Inventors: Lisa A. Butterick, Wilmington, DE (US); Darrin J. Pochan, Landenberg, PA (US); Joel P. Schneider, Newark, DE (US)

Correspondence Address: RATNERPRESTIA, P.O. BOX 1596, WILMINGTON, DE, 19899, US

(73) Assignee: University of Delaware, Newark, DE (US)

(21) Appl. No.: 12/407,079

(22) Filed: Mar. 19, 2009

Related U.S. Application Data
(60) Provisional application No. 61/037,996, filed on Mar. 19, 2008.

(51) Int. Cl. A61K 38/19; B05D 1/02; A61P 17/02

(52) U.S. Cl. 424/85.1; 427/427.4

(57) ABSTRACT

A method for delivering β-sheet peptide hydrogels to a target surface by shear-thinning and spraying the peptide hydrogel on the surface is provided. The β-sheet peptide hydrogels can be applied over a range of thicknesses and can cover broad surface areas. The β-sheet peptide hydrogels may also include a therapeutic agent.

### CROSS REFERENCE TO RELATED APPLICATIONS

This application claims benefit of priority to U.S. Provisional Application Ser. No. 61/037,996, filed Mar. 19, 2008, and which is incorporated herein, in its entirety, by reference.

### GOVERNMENT INTERESTS

This invention was made with government support under DE0163861, awarded by the National Institutes of Health. The government has certain rights in this invention.

### BACKGROUND OF THE INVENTION

Hydrogels are a class of materials that have significant promise for use in soft tissue and bone engineering and wound sealing, in part because of their well-hydrated, porous structure. For use in tissue regeneration, however, a material must be cyto-compatible, i.e., not toxic to the target cells, biocompatible, i.e., does not cause a significant immunological and inflammatory response in vivo and is preferably biodegradable, and have some rigidity.

To these ends, β-sheet peptide-based hydrogels that are capable of self-assembly in vivo or in vitro in response to environmental stimuli, such as pH, temperature, salt concentration, or specific ion concentrations, have been developed. In solution, the peptides are unfolded. Upon stimulation, the peptides first fold to form β-hairpins. The β-hairpin peptides then self-assemble to form β-sheet hydrogels. These peptide-based hydrogelation systems have been described in J. P. Schneider, et al., J Am Chem Soc 124: 15030-15037, 2002; D. J. Pochan, et al., J Am Chem Soc 125: 11802-11803, 2003; B. Ozbas, et al., Macromolecules 37: 7331-7337, 2004; K. Jajagopal and J. P. Schneider, Curr. Opin. Structural Biol. 14: 480-486, 2004; L. Haines-Butterick, et al., PNAS 104: 7791-7796, 2007. They are additionally described in US 2006/0025524 and US 2007/0128175, which are incorporated herein, in entirety, by reference.

When hydrogels are applied in vivo, the gel materials or precursor liquids do not remain localized to the site of application unless hydrogelation is rapid or the material is applied to a well-defined cavity. Therefore, a method is needed to apply hydrogels to tissue in such a way that the hydrogels remain localized to the site of application. In addition, methods for depositing thin hydrogel films on cell and tissue culture substrates and for depositing hydrogels over a broad surface, e.g., in wound sealing, are required.

### SUMMARY OF THE INVENTION

A method of delivering a preformed β-sheet hydrogel to a target site is provided, the method comprising shearing the hydrogel and spraying the sheared hydrogel onto the target site. Embodiments of the method include, but are not limited to, methods of spraying hydrogels comprising peptides selected from the group consisting of SEQ ID NOs: 1-69 to a target site. In a further embodiment, the hydrogel comprises a therapeutic agent.

### BRIEF DESCRIPTION OF THE DRAWINGS

FIG. 1. A schematic drawing of HPL8 in a folded β-hairpin conformation.

FIG. 2. Protocol for preparing and delivering a hydrogel as a spray. Cell culture medium is used as a stimulus to initiate self-assembly and hydrogelation. Shear is applied to thin the hydrogel and produce a low viscosity gel that can be delivered as a spray (shear-thin delivery). After spraying on the target surface, the hydrogel immediately recovers its mechanical properties and remains fixed at the site of application.

FIG. 3. Apparatus for delivery of a hydrogel as a spray. (a) Side view of an air brush apparatus used to deliver hydrogels as a spray, (b) Top view of a 1 wt % HPL8 (DMEM, 25 mM HEPES, pH 7.4) hydrogel formed in the top loading compartment of the air brush, (c-e) Top view of patterned hydrogel. Masks were laid over the petri dish, followed by delivery of a sheared, 1 wt % HPL8 gel as a spray. Plates were then stained with congo red (c,d) or fluorescein isothiocyanate (e).

FIG. 4. Circular dichroism of HPL8 spray gel. CD wavelength scan of a sheared 1 wt % HPL8 hydrogel shear delivered as a spray to a quartz cell. The ellipticity minimum at 218 nm is indicative of β-sheet structure within the sprayed hydrogel.

FIG. 5. LSCM image of sprayed gel. LSCM z-stack image (viewed along the y-axis) showing a sheared hydrogel (HPL8) delivered as a spray to the surface of a 4-well confocal plate. Gel was stained green with calcein for visualization (bottom of image is the surface of plate, top of image is the space above the gel). Scale bar is 50 μm.

FIG. 6. Cellular patterning with sprayed gel HPL8. (a) Protocol for patterning the attachment of cells to a surface. A mask is placed onto the surface of a polymer-coated glass slide, and shear-thinned hydrogel is sprayed onto the masked surface. Following application of the gel, the mask is removed and cells are allowed to attach to the surface. Cells attach preferentially to the hydrogel, creating a pattern of cells. (b) LSCM image of a single hydrogel spot on a glass slide to which cells have attached. A viability assay stained living cells green and dead cells red.

### DETAILED DESCRIPTION OF THE INVENTION

As described below, preformed β-sheet peptide hydrogels can be shear-thin delivered as a spray to provide broad surface coverage at target sites, such as tissue surfaces and tissue or cell-culture surfaces. Shear-thin delivery is achieved by using mechanical shear forces, for example, educting with gas or spraying through an aperture at high pressure, to thin the gel material allowing it to flow. Any spraying apparatus appropriate for delivering the hydrogel to a target surface in the desired amount and thickness may be used. In a preferred embodiment, the shearing and spraying of the hydrogel are substantially simultaneous. After delivery as a spray, the hydrogel remains localized at the site of application and retains its β-sheet structure. These peptide hydrogels can be deposited over a range of thickness from 1 μm to 1 mm, and can be used to pattern surfaces for site-selective cell attachment. Shear-thin delivery of the hydrogel as a spray allows broad surface coverage.

The hydrogel can also comprise a therapeutic agent and be utilized to deliver the therapeutic agent to a target site, such as to a tissue in vivo or in vitro. For example, the hydrogel may contain agents that stimulate cell proliferation or differentiation, stimulate wound healing, or inhibit bacterial growth. Such agents may include, but are not limited to analgesics, antibiotics, antineoplastics, hemostatic agents, anticoagulants, cytokines, growth factors, anti-inflammatories, small molecules, proteins, peptides, nucleotides, or cells. Spray delivery for β-sheet peptide hydrogels has broad medical application in, for example, tissue and bone engineering, regenerative and cosmetic treatment for hair and skin, cell-based diagnostics, surgery, wound-healing and wound-sealing. Spray delivery of β-sheet peptide hydrogels can also be used to apply gels to plastic or glass substrates for cell culture. The sprayed hydrogels may also be used in applying protective anti-bacterial coatings to a surface.

Examples of peptides that may be used in the practice of one or more aspect of the invention include, but are not limited to, the following:

| | | |
|---|---|---|
| HPL8 | VKVKVKVK V^{D}PPT KVEVKVKV | (SEQ. ID NO. 21) |
| MAX1 | VKVKVKVK V^{D}PPT KVKVKVKV | (SEQ. ID NO. 22) |
| MAX2 | VKVKVKVK V^{D}PPT KVKTKVKV | (SEQ. ID NO. 23) |
| MAX3 | VKVKVKTK V^{D}PPT KVKTKVKV | (SEQ. ID NO. 24) |
| MAX4 | KVKVKVKV K^{D}PPS VKVKVKVK | (SEQ. ID NO. 25) |
| MAX5 | VKVKVKVK V^{D}PPT KVKEKVKV | (SEQ. ID NO. 26) |
| MAX6 | VKVKVKVK V^{D}PPT KVKCKVKV | (SEQ. ID NO. 27) |
| MAX7 | VKVKVKVK V^{D}PGT KVKVKVKV | (SEQ. ID NO. 28) |
| MAX8 | VKVKVKVK VP^{D}PT KVKVKVKV | (SEQ. ID NO. 29) |
| MAX9 | VKVKVKVK VPPT KVKVKVKV | (SEQ. ID NO. 1) |
| MAX10 | VKVKVKVK V^{D}P^{D}PT KVKVKVKV | (SEQ. ID NO. 30) |
| MAX11 | VKVKKCK V^{D}PPT KVKCKVKV | (SEQ. ID NO. 31) |
| MAX12 | VKVKCKVK V^{D}PPT KVCVKVKV | (SEQ. ID NO. 32) |
| MAX13 | ISINYRTE I^{D}PPT SINYRTEI | (SEQ. ID NO. 33) |
| MAX14 | VKVKVCVK V^{D}PPT CVKVKVKV | (SEQ. ID NO. 34) |
| MAX15 | VKVKVCVK V^{D}PPT KVKVCVKV | (SEQ. ID NO. 35) |
| MAX16 | VKVKVKVC V^{D}PPT KVKVCVKV | (SEQ. ID NO. 36) |
| MAX17 | RGDVKVKVKVK V^{D}PPT KVKVKVKVR | (SEQ. ID NO. 37) |
| | GD | |
| MAX18 | VKVEVKVE V^{D}PPT KVEVKVEV | (SEQ. ID NO. 38) |
| MAX19 | VKVKVKVKVK V^{D}PPT KVKVKVKVKV | (SEQ. ID NO. 39) |
| MAX20 | VKVKVKVK YNGT KVKVKVKV | (SEQ. ID NO. 2) |
| MAX21 | VKVKVK V^{D}PPT KVKVKV | (SEQ. ID NO. 40) |
| MAX22 | VKVKVKVK GGGG KVKVKVKV | (SEQ. ID NO. 3) |
| MAX23 | VEVEVEVE V^{D}PPT EVEVEVEV | (SEQ. ID NO. 41) |
| MAX24 | VXVXVXVX V^{D}PPT XVXVXVXV | (SEQ. ID NO. 42) |
| | X = Ornithine | |
| MAX25 | VXVXVXVX V^{D}PPT XVXVXVXV | (SEQ. ID NO. 43) |
| | X = Diaminobutyric acid | |
| MAX26 | VXVXVXVX V^{D}PPT XVXVXVXV | (SEQ. ID NO. 44) |
| | X = Diaminopropionic acid | |
| MAX27 | VYXYXYX Y^{D}PPT XYXYXYXY | (SEQ. ID NO. 45) |
| | X = Valine | |
| MAX28 | VRVRVRVR V^{D}PPT RVRVRVRV | (SEQ. ID NO. 46) |
| MAX29 | VKVKVKVKRGDKVKVKVKV | (SEQ. ID NO. 4) |
| MAX30 | XKXKXKXK V^{D}PPT KXKXKXKX | (SEQ. ID NO. 47) |
| | X = Aminoisobutyric acid | |
| MAX31 | XKXKXKXK V^{D}PPT KXKXKXKX | (SEQ. ID NO. 48) |
| | X = Norvaline | |
| MAX32 | XKXKXKXK V^{D}PPT KXKXKXKX | (SEQ. ID NO. 49) |
| | X = Norleucine | |
| MAX33 | FKFKFKFK V^{D}PPT KFKFKFKF | (SEQ. ID NO. 50) |
| MAX34 | IKIKIKIK V^{D}PPT KIKIKIKI | (SEQ. ID NO. 51) |
| MAX35 | HWSFTIKIT V^{D}PPT HWSFTIKIT | (SEQ. ID NO. 52) |

In addition to the amino acids specifically recited above, at any position of any of the above peptides indicated with X, each X can independently be any natural or non-natural amino acid (L or D stereochemistry) or any analog of an amino acid known to those skilled in the art. In this application, D stereochemistry will be indicated by a superscript before the D amino acid, thus ^{D}P is D-proline.

In some embodiments of the invention, peptides may fit the general formula VKVKVKVK(XXXX)ₐKVKVKV(XXXX)_{b}KVKVKVKV (SEQ ID NO:5). Each of these peptides adopts a 3-stranded β-sheet conformation. (Rughani, et al., Biomacromolecules, accepted Mar. 4, 2009). Specific examples of 3-stranded β-sheet forming peptides include, but are not limited to,

| | | | |
|---|---|---|---|
| MAX36 | (XXXX)ₐ = V^{D}PPT | (XXXX)^{b} = K^{D}PPK | (SEQ. ID NO. 53) |
| MAX37 | (XXXX)ₐ = V^{D}PGT | (XXXX)_{b} = K^{D}PGK | (SEQ. ID NO. 54) |
| MAX38 | (XXXX)ₐ = D^{D}PGT | (XXXX)_{b} = K^{D}PPK | (SEQ. ID NO. 55) |
| MAX39 | (XXXX)ₐ = V^{D}PAT | (XXXX)_{b} = K^{D}PAK | (SEQ. ID NO. 56) |
| MAX40 | (XXXX)ₐ = V^{D}PPT | (XXXX)_{b} = K^{D}PGK | (SEQ. ID NO. 57) |
| MAX41 | (XXXX)ₐ = V^{D}PPT | (XXXX)_{b} = KNGK | (SEQ. ID NO. 6) |
| MAX42 | (XXXX)ₐ = VNGT | (XXXX)_{b} = K^{D}PPK | (SEQ. ID NO. 7) |
| MAX43 | (XXXX)ₐ = VNGT | (XXXX)_{b} = KNGK | (SEQ. ID NO. 58) |
| MAX44 | (XXXX)ₐ = V^{D}PAT | (XXXX)_{b} = K^{D}P^{D}AK | (SEQ. ID NO. 59) |

In addition to the amino acids specifically recited above, at any position of any of the above peptides indicated with X, each X can independently be any natural or non-natural amino acid (L or D stereochemistry) or any analog of an amino acid known to those skilled in the art. Preferably, each (XXXX)ₐ and (XXXX)_{b} may comprise a sequence capable of forming a turn (e.g., a β-turn).

In some embodiments of the invention, peptides may fit the following general formulas:

| | | | |
|---|---|---|---|
| MAXX₁ | (VK)ₘ V^{D}PPT (KV)ₙ | m = 1-100, n = 1-100 | (SEQ. ID NO. 60) |
| MAXX₂ | (VK)ₘ VPPT (KV)ₙ | m = 1-100, n = 1-100 | (SEQ. ID NO. 8) |
| MAXX₃ | (VK)ₘ V^{D}P^{D}PT (KV)ₙ | m = 1-100, n = 1-100 | (SEQ. ID NO. 61) |
| MAXX₄ | (VK)ₘ GGGG (KV)ₙ | m = 1-100, n = 1-100 | (SEQ. ID NO. 9) |
| MAXX₅ | (VK)ₘ VP^{D}PT (KV)ₙ | m = 1-100, n = 1-100 | (SEQ. ID NO. 62) |
| MAXX₆ | (VK)ₘ YNGT (KV)ₙ | m = 1-100, n = 1-100 | (SEQ. ID NO. 10) |
| MAXX₇ | (VK)ₘ VRGD (KV)ₙ | m = 1-100, n = 1-100 | (SEQ. ID NO. 11) |

wherein each m and n may independently be from 1-100 and m may or may not equal n;

| | |
|---|---|
| VKVKVKVKVDPPTKVKVKVKV-NH₂ | (SEQ ID NO:63) |
| VKVKVKVKVDPPTKVKVKVKV-NH₂ | (SEQ ID NO:64) |

wherein Na-butylated lysine residues are present at the bold positions;

| | |
|---|---|
| VK(VK)ₘVKVKVDPPTKVKV(KV)ₙKV-NH₂, | (SEQ ID NO:65) |

where m=1-20 and n=1-20 and m may be the same or different as n in any given peptide.

In some embodiments, one or more amino acids of the turn region may be substituted and/or modified as compared to the turn region of MAX1. In some embodiments, turn sequences may be incorporated that not only play a structural role but also play a biofunctional role. For example, RGD (SEQ ID NO:66) binding epitopes are normally found within turn regions of proteins known to be important in cell adhesion events, and residues that flank RGD provide additional specificity to the binding event. Incorporating these epitopes into the turn regions of self-assembling hairpins may lead to hydrogel scaffolds having enhanced cell adhesion properties.

### EXAMPLES

### 1. Preparation of Hydrogel

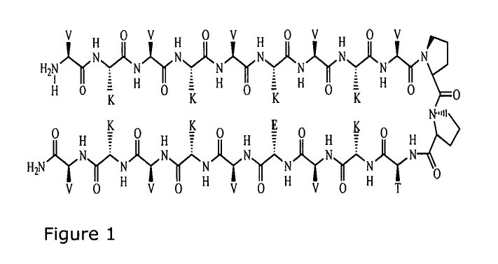

The peptide, HPL8 (SEQ ID NO: 21), in the folded state, is a 20 amino acid β-hairpin comprised of β-strands of alternating valine and lysine residues flanking a type II′ turn (FIG. 1). When dissolved in an aqueous buffered solution (25 mM HEPES, pH 7.4), HPL8 unfolds. Solutions of the unfolded peptide are free-flowing. When an equal volume of cell culture media (DMEM, pH 7.4) is added to a solution of unfolded peptide, the peptide folds into an amphiphilic β-hairpin. Once in the folded state, HPL8 self-assembles to form a rigid microporous hydrogel composed of non-covalently cross-linked β-sheet rich fibrils (Haines-Butterick, et al., PNAS USA 2007, 104, 7791-7796).

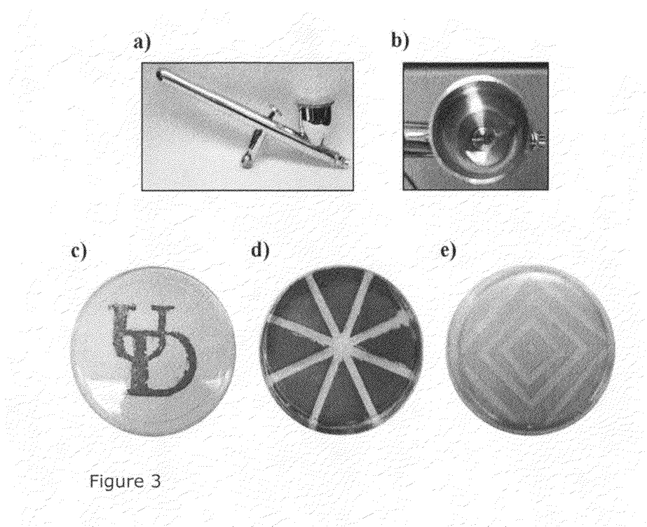

An HPL8 hydrogel was prepared by adding 500 μL of 25 mM HEPES, pH 7.4 to a vial containing 10 mg of HPL8 peptide, giving rise to a soluble 2 wt % HPL8 peptide solution. An equal volume of DMEM supplemented with 25 mM HEPES, pH 7.4 was added to the soluble 2 wt % HPL8 peptide solution and the mixture was immediately transferred to a 15 ml gravity-feed cup on top of an Iwata Revolution CR airbrush equipped with a 0.5 mm screw-in nozzle (FIG. 3(a,b)). Gelation occurred within about 1 minute, after which time, the gels were allowed to stiffen for 15 to 60 minutes. This procedure yielded 1 mL of a 1 wt % HPL8 hydrogel.

### 2. Shear-Thin Delivery of HPL8 Gel as a Spray.

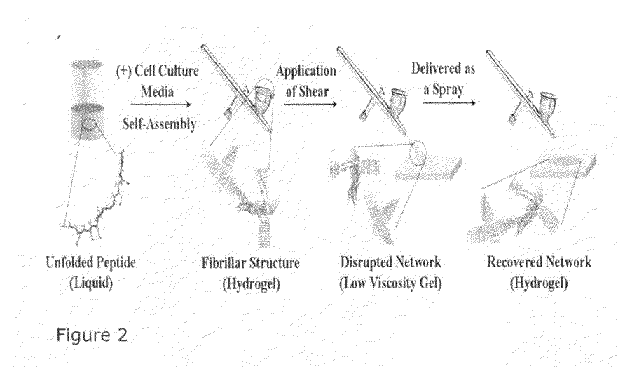

The airbrush described in Example 1 was connected to a nitrogen tank equipped with a regulator set to a range of approximately 10 psi to approximately 20 psi. Nitrogen (N2) was passed through the airbrush to provide a shear force that disrupted the non-covalently cross-linked network of the hydrogel in the gravity-feed cup. Pulling back on the airbrush lever exposed the gel in the gravity feed cup to the nitrogen gas below, which flows through the brush. The flowing gas provided suction to pull the gel into the flowing gas and consequently shear-thinned the gel into particles that were sprayed through the brush nozzle with the exiting gas. Thus, the shear-thinning procedure produces gel particles that can be sprayed through the airbrush and onto a surface. Using this method, the shearing and spraying of the hydrogel are substantially simultaneous. The mist of hydrogel produced immediately recovered rigidity upon contact with the surface and remained fixed on the surface at the site of application. Hydrogels remained fixed at the site of application, even when submerged in water and agitated. The shear-thin and spraying protocol is shown in FIG. 2.

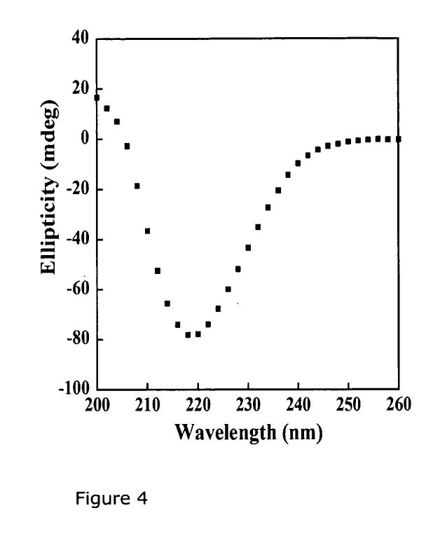

CD wavelength spectra were collected on a Jasco J-810 spectropolarimeter employing a 0.01 mm detachable quartz cell. HPL8 gels (1 wt %) were prepared as described in Example 1 and shear-thin delivered via the airbrush to the detachable quartz cell. Measurements were taken immediately after shear-thin delivery. Ellipticity in millidegrees was monitored from 260 nm to 200 nm at 37° C. using a step size of 2 nm. The CD wavelength spectrum, shown in FIG. 4, is characteristic of β-sheet rich structure with a minimum at 218 nm. This data demonstrates that the secondary structure of the peptide is not affected by the shearing or spray delivery procedures. In addition, the hydrogels shown in FIG. 3(c and d) bound congo-red dye, indicating that the β-sheet structure of the fibrils is also unaffected.

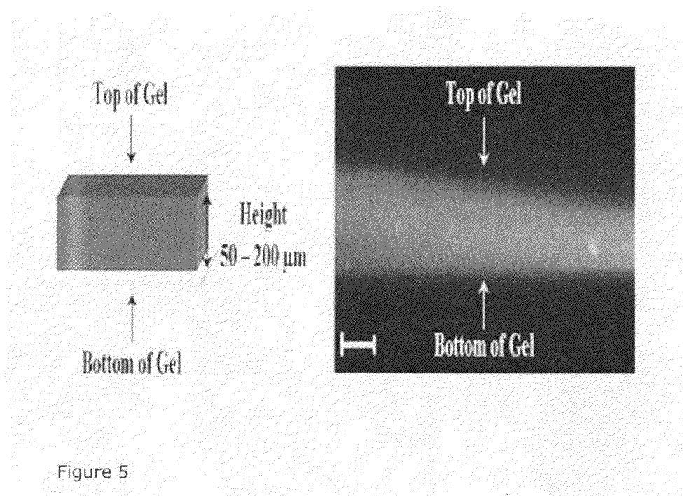

An LSCM (laser scan confocal microscope) z-stack image of a 1 wt % HPL8 hydrogel sprayed onto the surface of one well of an 8-well confocal plate and stained with calcein for visualization is shown in FIG. 5. The z-stack image was generated by taking xy slices starting below the glass slide and up into the space above the gel. The slices were combined to form a z-stacked image (viewing perpendicular to the z-axis), where the bottom of the image is the region beneath the slide and the top of the image is the region above the gel. Images were taken using a 10× magnification on a Zeiss 510 LSCM microscope.

The height of the sprayed hydrogel on the surface can be as thin as approximately 1 μm. The height of the hydrogel can be adjusted to any desired height by manipulating the rate of gel delivery within the spray brush and by using multiple, consecutive sprays.

4. Patterning with the Sprayed Hydrogel.

Hydrogels can be sprayed in patterns by laying a mask on the surface to be sprayed. FIG. 3(c-e) shows masked surfaces of Petri dishes after spraying with an HPL8 hydrogel as described in Examples 1 and 2. Tape was used to mask the desired portions of the dish. The 1 wt % HPL8 gel was shear-thin delivered via the airbrush to the entire dish and allowed to set for about 5 minutes. The tape was slowly removed leaving behind a thin layer of HPL8 gel, coating only on the surface of the Petri dish that was not masked with tape. To aid in visualization, the gels were stained for about 10 min with either a solution of congo red (FIG. 3c,d), which binds to β-sheet structure, or a solution of fluorescein isothiocyanate (FIG. 3e), which covalently functionalizes the free amines of the peptide.

Hydrogel sprays can also be used to create cell patterns on surfaces. Patterned surfaces were generated by first spin-coating microscope slides with a triblock comb polymer (61 wt % (methyl methacrylate (MMA), 21 wt % hydroxyl poly(oxyethylene) methacrylate (HPOEM), and adding 18 wt % poly(ethylene glycol) methyl ether methacrylate (POEM)) to the slides. The slides were centrifuged at about 2500 rpm for 20 seconds then cured at 60° C. under vacuum overnight. Cells are unable to attach to the polymer-coated slides. A mask containing small holes of about 2 mm in diameter was placed on top of the polymer-coated slides, and HPL8 hydrogel was then sprayed through the mask. The mask was removed leaving behind a pattern of small islands of hydrogel, each about 2 mm in diameter, surrounded the by polymer.

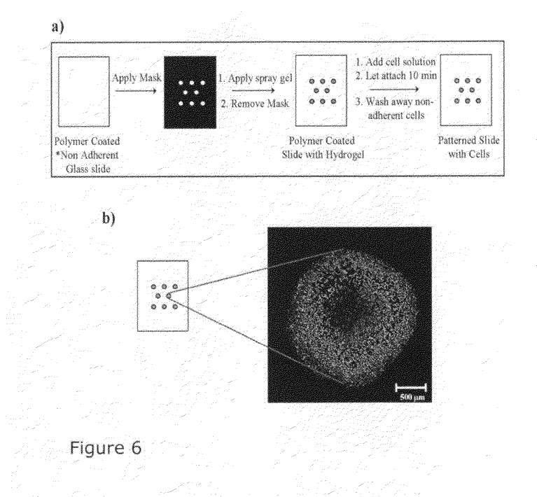

A solution of 5×10⁶ C3H10t1/2 cells/mL, suspended in DMEM cell culture medium, was prepared and 2 ml of this solution was added to the surface of the patterned slide and incubated for 5 minutes. The slide was then washed in DMEM to remove unattached cells. A live/dead assay (Molecular Probes) was performed to determine cellular attachment and viability and to visualize the location of cells. With this assay, live cells fluoresce green and dead cells fluoresce red. FIG. 6 shows that cells attached only on the hydrogel-coated areas of the slide. The majority of the cells were viable, indicating that after shear-thin delivery as a spray, the HPL8 hydrogel is not cytotoxic.

Although the invention is illustrated and described herein with reference to specific embodiments, the invention is not intended to be limited to the details shown. Rather, various modifications may be made in the details within the scope and range of equivalents of the claims and without departing from the invention.

What is claimed is:

1. A method of delivering a preformed β-sheet peptide hydrogel to a target site, the method comprising shearing the hydrogel and spraying the sheared hydrogel onto the target site.

2. The method according to claim 1, wherein the β-sheet peptide hydrogel comprises β-hairpin peptides.

3. The method according to claim 2, wherein the hydrogel comprises peptides selected from the group consisting of SEQ ID NOs: 1-69.

4. The method according to claim 3, wherein the hydrogel comprises a therapeutic agent.

5. The method according to claim 4, wherein the therapeutic agent is selected from the group consisting of analgesics, antibiotics, antineoplastics, hemostatic agents, anticoagulants, cytokines, growth factors, anti-inflammatories, cells, small molecules, nucleotides, proteins and peptides.

6. The method of claim 1, wherein the target site is a surface for cell-culture or tissue-culture.

7. The method according to claim 6, wherein the surface is glass or plastic.

8. The method according to claim 7, wherein the hydrogel comprises peptides selected from the group consisting of SEQ ID NOs: 1-69.

9. The method according to claim 6, wherein the hydrogel comprises a therapeutic agent.

10. The method according to claim 9, wherein the therapeutic agent is selected from the group consisting of analgesics, antibiotics, antineoplastics, hemostatic agents, anticoagulants, cytokines, growth factors, anti-inflammatories, cells, small molecules, nucleotides, proteins and peptides.

11. The method of claim 1, wherein the target site is a biological tissue.

12. The method according to claim 11, wherein the hydrogel comprises peptides selected from the group consisting of SEQ ID NOs: 1-69.

13. The method according to claim 11, wherein the hydrogel comprises a therapeutic agent.

14. The method according to claim 13, wherein the therapeutic agent is selected from a group consisting of analgesics, antibiotics, antineoplastics, hemostatic agents, anticoagulants, cytokines, growth factors, anti-inflammatories, cells, small molecules, nucleotides, proteins and peptides.

15. The method according to claim 11, wherein the tissue is human tissue.

16. The method according to claim 11, wherein the tissue is liver, lung or heart tissue.

17. The method according to claim 11, wherein the tissue is wound tissue.

18. The method according to claim 1, wherein the hydrogel comprises the peptide of SEQ ID NO:21.

19. The method according to claim 6, wherein the hydrogel comprises the peptide of SEQ ID NO:21.

20. The method according to claim 11, wherein the hydrogel comprises the peptide of SEQ ID NO:21.

21. The method according to claim 1, wherein shearing the hydrogel and spraying the sheared hydrogel onto the target site are substantially simultaneous.

22. A method of delivering a preformed β-sheet peptide hydrogel to a target site, the method comprising shearing the hydrogel and spraying the sheared hydrogel onto the target site, wherein the sprayed hydrogel remains localized at the target site and retains the β-sheet structure.


SEQUENCE LISTING (69 sequences)

SEQ ID NO: 1 - protein, 20 aa, Artificial sequence
  synthetic peptide
VKVKVKVKVPPTKVKVKVKV

SEQ ID NO: 2 - protein, 20 aa, Artificial sequence
  synthetic peptide
VKVKVKVKYNGTKVKVKVKV

SEQ ID NO: 3 - protein, 20 aa, Artificial sequence
  synthetic peptide
VKVKVKVKGGGGKVKVKVKV

SEQ ID NO: 4 - protein, 20 aa, Artificial sequence
  synthetic peptide
VKVKVKVKVRGDKVKVKVKV

SEQ ID NO: 5 - protein, 30 aa, Artificial sequence
  synthetic peptide
VKVKVKVKXXXXKVKVKVXXXXKVKVKVKV

SEQ ID NO: 6 - protein, 30 aa, Artificial sequence
  synthetic peptide
VKVKVKVKVPPTKVKVKVKNGKKVKVKVKV

SEQ ID NO: 7 - protein, 30 aa, Artificial sequence
  synthetic peptide
VKVKVKVKVNGTKVKVKVKPPKKVKVKVKV

SEQ ID NO: 8 - protein, 8 aa, Artificial sequence
  synthetic peptide
VKVPPTKV

SEQ ID NO: 9 - protein, 8 aa, Artificial sequence
  synthetic peptide
VKGGGGKV

SEQ ID NO: 10 - protein, 8 aa, Artificial sequence
  synthetic peptide
VKYNGTKV

SEQ ID NO: 11 - protein, 8 aa, Artificial sequence
  synthetic peptide
VKVRGDKV

SEQ ID NO: 12 - protein, 20 aa, Artificial sequence
  synthetic peptide
VKVKVKVKXXXXKVKVKVKV

SEQ ID NO: 13 - protein, 20 aa, Artificial sequence
  synthetic peptide
VKVKVKVKVGGTKVKVKVKV

SEQ ID NO: 14 - protein, 30 aa, Artificial sequence
  synthetic peptide
VKVKVKVKVDRKADGYIDFEEVKVKVKVKV

SEQ ID NO: 15 - protein, 6 aa, Artificial sequence
  peptide epitope
TRGDSP

SEQ ID NO: 16 - protein, 4 aa, Artificial sequence
  peptide epitope
RGDG

SEQ ID NO: 17 - protein, 4 aa, Artificial sequence
  peptide epitope
RGDY

SEQ ID NO: 18 - protein, 4 aa, Artificial sequence
  peptide epitope
RGDW

SEQ ID NO: 19 - protein, 7 aa, Artificial sequence
  epitope
FHRRIKA

SEQ ID NO: 20 - protein, 7 aa, Artificial sequence
  epitope
GRGDSPY

SEQ ID NO: 21 - protein, 20 aa, Artificial sequence
  synthetic peptide
VKVKVKVKVPPTKVEVKVKV

SEQ ID NO: 22 - protein, 20 aa, Artificial sequence
  synthetic peptide
VKVKVKVKVPPTKVKVKVKV

SEQ ID NO: 23 - protein, 20 aa, Artificial sequence
  synthetic peptide
VKVKVKVKVPPTKVKTKVKV

SEQ ID NO: 24 - protein, 20 aa, Artificial sequence
  synthetic peptide
VKVKVKTKVPPTKVKTKVKV

SEQ ID NO: 25 - protein, 20 aa, Artificial sequence
  synthetic peptide
KVKVKVKVKPPSVKVKVKVK

SEQ ID NO: 26 - protein, 20 aa, Artificial sequence
  synthetic peptide
VKVKVKVKVPPTKVKEKVKV

SEQ ID NO: 27 - protein, 20 aa, Artificial sequence
  synthetic peptide
VKVKVKVKVPPTKVKCKVKV

SEQ ID NO: 28 - protein, 20 aa, Artificial sequence
  synthetic peptide
VKVKVKVKVPGTKVKVKVKV

SEQ ID NO: 29 - protein, 20 aa, Artificial sequence
  synthetic peptide
VKVKVKVKVPPTKVKVKVKV

SEQ ID NO: 30 - protein, 20 aa, Artificial sequence
  synthetic peptide
VKVKVKVKVPPTKVKVKVKV

SEQ ID NO: 31 - protein, 19 aa, Artificial sequence
  synthetic peptide
VKVKKCKVPPTKVKCKVKV

SEQ ID NO: 32 - protein, 20 aa, Artificial sequence
  synthetic peptide
VKVKCKVKVPPTKVCVKVKV

SEQ ID NO: 33 - protein, 20 aa, Artificial sequence
  synthetic peptide
ISINYRTEIPPTSINYRTEI

SEQ ID NO: 34 - protein, 20 aa, Artificial sequence
  synthetic peptide
VKVKVCVKVPPTCVKVKVKV

SEQ ID NO: 35 - protein, 20 aa, Artificial sequence
  synthetic peptide
VKVKVCVKVPPTKVKVCVKV

SEQ ID NO: 36 - protein, 20 aa, Artificial sequence
  synthetic peptide
VKVKVKVCVPPTKVKVCVKV

SEQ ID NO: 37 - protein, 26 aa, Artificial sequence
  synthetic peptide
RGDVKVKVKVKVPPTKVKVKVKVRGD

SEQ ID NO: 38 - protein, 20 aa, Artificial sequence
  synthetic peptide
VKVEVKVEVPPTKVEVKVEV

SEQ ID NO: 39 - protein, 24 aa, Artificial sequence
  synthetic peptide
VKVKVKVKVKVPPTKVKVKVKVKV

SEQ ID NO: 40 - protein, 16 aa, Artificial sequence
  synthetic peptide
VKVKVKVPPTKVKVKV

SEQ ID NO: 41 - protein, 20 aa, Artificial sequence
  synthetic peptide
VEVEVEVEVPPTEVEVEVEV

SEQ ID NO: 42 - protein, 20 aa, Artificial sequence
  synthetic peptide
VXVXVXVXVPPTXVXVXVXV

SEQ ID NO: 43 - protein, 20 aa, Artificial sequence
  synthetic peptide
VXVXVXVXVPPTXVXVXVXV

SEQ ID NO: 44 - protein, 20 aa, Artificial sequence
  synthetic peptide
VXVXVXVXVPPTXVXVXVXV

SEQ ID NO: 45 - protein, 19 aa, Artificial sequence
  synthetic peptide
VYVYVYVYPPTVYVYVYVY

SEQ ID NO: 46 - protein, 20 aa, Artificial sequence
  synthetic peptide
VRVRVRVRVPPTRVRVRVRV

SEQ ID NO: 47 - protein, 20 aa, Artificial sequence
  synthetic peptide
XKXKXKXKVPPTKXKXKXKX

SEQ ID NO: 48 - protein, 20 aa, Artificial sequence
  synthetic peptide
XKXKXKXKVPPTKXKXKXKX

SEQ ID NO: 49 - protein, 20 aa, Artificial sequence
  synthetic peptide
XKXKXKXKVPPTKXKXKXKX

SEQ ID NO: 50 - protein, 20 aa, Artificial sequence
  synthetic peptide
FKFKFKFKVPPTKFKFKFKF

SEQ ID NO: 51 - protein, 20 aa, Artificial sequence
  synthetic peptide
IKIKIKIKVPPTKIKIKIKI

SEQ ID NO: 52 - protein, 22 aa, Artificial sequence
  synthetic peptide
HWSFTIKITVPPTHWSFTIKIT

SEQ ID NO: 53 - protein, 30 aa, Artificial sequence
  synthetic peptide
VKVKVKVKVPPTKVKVKVKPPKKVKVKVKV

SEQ ID NO: 54 - protein, 30 aa, Artificial sequence
  synthetic peptide
VKVKVKVKVPGTKVKVKVKPGKKVKVKVKV

SEQ ID NO: 55 - protein, 30 aa, Artificial sequence
  synthetic peptide
VKVKVKVKVPGTKVKVKVKPPKKVKVKVKV

SEQ ID NO: 56 - protein, 30 aa, Artificial sequence
  synthetic peptide
VKVKVKVKVPATKVKVKVKPAKKVKVKVKV

SEQ ID NO: 57 - protein, 30 aa, Artificial sequence
  synthetic peptide
VKVKVKVKVPPTKVKVKVKPGKKVKVKVKV

SEQ ID NO: 58 - protein, 30 aa, Artificial sequence
  synthetic peptide
VKVKVKVKVNGTKVKVKVKNGKKVKVKVKV

SEQ ID NO: 59 - protein, 30 aa, Artificial sequence; identical to SEQ ID NO: 56
  synthetic peptide

SEQ ID NO: 60 - protein, 8 aa, Artificial sequence
  synthetic peptide
VKVPPTKV

SEQ ID NO: 61 - protein, 8 aa, Artificial sequence
  synthetic peptide
VKVPPTKV

SEQ ID NO: 62 - protein, 8 aa, Artificial sequence
  synthetic peptide
VKVPPTKV

SEQ ID NO: 63 - protein, 20 aa, Artificial sequence
  synthetic peptide
VKVKVKVKVPPTKVKVKVKV

SEQ ID NO: 64 - protein, 20 aa, Artificial sequence
  synthetic peptide
VKVKVKVKVPPTKVKVKVKV

SEQ ID NO: 65 - protein, 20 aa, Artificial sequence
  synthetic peptide
VKVKVKVKVPPTKVKVKVKV

SEQ ID NO: 66 - protein, 3 aa, Artificial sequence
  binding epitope
RGD

SEQ ID NO: 67 - protein, 12 aa, Artificial sequence
  calcium binding loop within the EF hand domain of Troponin C
DRKADGYIDFEE

SEQ ID NO: 68 - protein, 3 aa, Artificial sequence
  epitope
SDV

SEQ ID NO: 69 - protein, 3 aa, Artificial sequence
  epitope
RNS